(19) United States
(12) Patent Application Publication
(10) Pub. No.: US 2026/0306515 A1
(43) Pub. Date: Oct. 8, 2026

(54) FRUIT-BASED FREEZE-DRIED EDIBLES

(71) Applicant: Daniel E. Wente, Cincinnati, OH (US)

(72) Inventor: Daniel E. Wente, Cincinnati, OH (US)

(21) Appl. No.: 19/169,544

(22) Filed: Apr. 3, 2025

(51) Int. Cl. A61K 31/00; A23F 5/38; A23F 5/40; A61K 9/00; A61K 36/07; A61K 36/185; A61K 36/31; A61K 36/41; A61K 36/815; A61K 47/14; A61K 47/44; A61K 47/46

(52) U.S. Cl. CPC A61K 31/658; A23F 5/385; A23F 5/40; A61K 9/0056; A61K 36/07; A61K 36/31; A61K 36/3482; A61K 36/41; A61K 36/815; A61K 47/14; A61K 47/44; A61K 47/46

(57) ABSTRACT

Freeze-dried, lipid modified, fruit-based edibles in a form that can rapidly melt in the mouth and be absorbed through the oral mucosa of a subject. The composition can preferably include a pleasant tasting fruit puree, at least one nutraceutical, such as A-8-THC and/or CBD extracted from hemp, and an edible oil which can be combined with an edible oil/wax, in which the composition is freeze-dried into a ready to be consumed product form which melts in the mouth. While intended to be pleasant in taste, the inventive pucks or pellets can be stored in child-resistant packaging or containers and are intended to be molded in shapes that appear medicinal in nature, and are not readily identifiable to children as a type of treat, or candy.

### FIELD OF THE INVENTION

The present invention relates in general to compositions and methods for delivering a therapeutic agent, and in particular to a freeze-dried, fruit-based edible for delivering cannabinoids and other supplements in a form that instantly melts in the mouth.

### BACKGROUND OF THE INVENTION

This invention is intended to provide nutraceuticals that are useful for the maintenance and support of brain function, the heart and circulatory system, healthy joints, skin, eyes, and overall general health. Nutraceuticals are natural, bioactive chemical compounds that provide physiological benefits beyond basic nutrition. For example, many nutraceuticals are widely recognized for treating joint and spine problems, such as chondroitin sulfate, fish oils, and glucosamine sulfate. There are a number of nutraceuticals sold over the counter as all-natural means for boosting immune function, curing bodily ailments, and enhancing mental function. Common examples of nutraceuticals include vitamins, minerals, herbal extracts, and probiotics; functional foods, such as fortified cereals, omega-3 enriched eggs, and probiotic-rich foods, and other products such as protein bars and powders, and energy drinks that include herb extracts such as ephedra, Ginko biloba, goldenseal, valerian, ginseng, green tea extract, echinacea, CBD/THC, fungi, collagen, hyaluronic acid, caffeine, and dietary fibers.

Cannabis plants include both hemp and marijuana plants, both of which produce cannabinoids, a group of biologically active chemicals which produce mental and physical effects when consumed. It is a common misconception that hemp and cannabis are two different species of plant, but in fact hemp and cannabis are the same plant. The major difference is that hemp is a mixture of male and female cannabis plants, and its flowers produce less than 0.3% THC. Hemp plants also tend to be taller and contain more fiber in their stalks. For this reason, hemp plants are often used to make textiles.

While science doesn't differentiate between hemp and marijuana cannabis plants, the law does. Legally, the key difference between the two is tetrahydrocannabinol (THC) content. The term “hemp” is used to mean cannabis that contains 0.3% or less THC content by dry weight. Because the THC level in hemp is so low, it's unlikely to cause intoxication. In contrast, “marijuana” refers to cannabis that has more than 0.3% THC by dry weight.

Cannabinoid receptors (i.e., CB₁ and CB₂ receptors) are located throughout the body and are part of the endocannabinoid system, which is involved in a variety of physiological processes, and which continues to receive attention as a therapeutic target. Broad claims for marijuana and its derivatives are being made, but these remain to be proven. The CBI receptor is particularly abundant in the central nervous system (CNS), mainly the brain and to some extent in peripheral tissues, while the expression of CB₂ receptors is generally restricted to the brainstem and the hippocampal neurons centrally, and peripherally in circulating immune cells, the spleen, osteocytes, osteoclasts, and hepatic macrophage cells. However, CB₂ expression is highly inducible in the CNS following inflammation or injury.

Cannabinoids mimic the action of anandamide, a naturally occurring neurotransmitter which is synthesized in areas of the brain controlling memory, motivation, higher thought processes, and movement control. Anandamide can also affect one's appetite and reaction to pain, may slow cancer cell proliferation, and exhibit both anti-anxiety and antidepressant properties. While anandamide, like all neurotransmitters, is metabolized quickly in the body, cannabinoids can bind to the same receptors as anandamide. This group of biologically active cannabinoids can include delta-9-tetrahydrocannabinol (Δ-9-THC), cannabidiol (CBD), Δ-8-THC, cannabigerol (CBG), cannabinol (CBN), tetrahydrocannabivarin (THCV), Cannabichromene (CBC), cannabivarin (CBDV), Delta-9-tetrahydrocannabiphoral (THCP), cannabidiphorol (CBDP), Δ-6-THC, Δ-10-THC, Cannabidiolic acid (CBDA), Tetrahydrocannabinolic acid (THCA), 11-hydroxy-Δ-9-THC, levonantradol, among others.

Δ-9-THC is the active ingredient in FDA-approved dronabinol (Marinol and Syndros), and a synthetic Δ-9-THC is also FDA-approved under the name nabilone (Cesamet). These medications are approved for specific conditions such as HIV/AIDS-induced anorexia (AIDS wasting syndrome), epilepsy, neuropathic pain, and treatment of spasticity associated with multiple sclerosis. Δ-9-THC has also been used to treat or prevent chemotherapy-induced nausea and vomiting, especially when other medications do not work.

CBD is typically derived from hemp because of hemp's low THC concentration; it is non-psychoactive and has been marketed in gummies, lotions, oils, lollipops, vapes, pet products, and even toothpaste. It's been touted as a treatment for inflammation, anxiety, insomnia, pain, PTSD, and depression, and it's becoming increasingly popular. In 2022, the CBD market was valued at $18 billion, and it's expected to triple in the next 10 years.

Δ-8 -THC is known to bind to brain receptors that control bodily functions such as sleep and memory, and is typically synthesized from cannabidiol (CBD), which is extracted from hemp, because natural quantities of Δ-8-THC found in hemp (or marijuana) plants are low. While both Δ-9-THC and Δ-8-THC are forms of THC, when people refer to “THC” they usually mean the Δ-9-THC that's found in high concentrations in marijuana plants. While Δ-8-THC doesn't produce intoxication or the intense “high” of Δ-9-THC, it can affect mood, thoughts, feelings, and behavior. Other common Δ-9-THC side effects such as paranoia, anxiety, and drowsiness are also less potent with Δ-8-THC. Indeed, Δ-8-THC is often referred to as “marijuana lite” or “diet weed.”

The 2018 Farm Bill made it legal to grow hemp, or cannabis containing less than 0.3% THC, throughout the United States. It also made hemp-derived CBD products federally legal. In addition to the Δ-9-THC containing products dronabinol and nabilone, the FDA has approved one CBD product: Epidiolex. Epidiolex is for patients 2 years and older with seizure problems, namely Dravet syndrome (DS) or Lennox-Gastaut syndrome (LGS). Although the exact mechanism of action is unknown, both CBD and THC are thought to be efficacious in their role for pediatric epilepsy. Using the non-psychoactive component, CBD, allows patients to have few side psychoactive side effects while still having the benefit of reduction in seizure frequency.

While the FDA has not approved any other cannabis-derived products, markets are booming in states that have legalized recreational and medicinal marijuana use. With all this forward movement, more and more companies are looking to cash in. While cannabis containing more than 0.3% THC is not federally legal, state laws can vary. To do business in these states, companies must follow the various regulations of the market. Hemp oil is federally legal and contains less than or equal to 0.3% THC. It can be bought over the counter at pharmacies, grocery stores, and even gas stations. Some states allow patients or caregivers to possess CBD products greater than 5% with a state permit. However, it is important to recognize that none of these products are FDA approved.

Typically, nutraceutical and other dietary supplements employ the oral administration route, which involves putting a pill or tablet into the mouth and swallowing it. However, one of the downsides of oral administration is that it can be inefficient. The digestive system and liver start to break down drugs via this route before they can reach the bloodstream, such that the final, active concentration of the drug can be reduced. This is known as the “first pass effect.” As a result, delivery through the oral mucosa can be a useful alternative, and provide numerous advantages over the other delivery routes such as oral, parenteral and dermal, due to its rich blood supply, rapid onset of action, avoidance of the first pass metabolism, and lack of enzymatic degradation.

Recently, products commonly referred to as fast-melt, rapid-disintegrating, and/or freeze-dried formulations, as well as orally disintegrating tablets (ODTs) and films have gained more attention, along with conventional sublingual and buccal formulations, especially for people who have difficulty swallowing or those with digestion issues. Such orodispersible compositions typically require less than a minute to dissolve in the mouth, and the active ingredient is released and absorbed through pre-gastric (oral mucosa, pharynx, and esophagus) as well as gastric means. Recent market studies indicate that more than half of the patient population prefers such orally disintegrating dosage forms This direct route offers a high bioavailability compared to other means, and allows the active ingredient to bypass the digestive system and liver metabolism. Administration is simple and painless, and the effects are experienced more quickly, making them a favored choice for those looking for rapid relief.

However, while sublingual, buccal and other orodispersible formulations are useful for being absorbed through the oral mucosa to maximize bioavailability, they generally have an unacceptable taste. Indeed, the sides and bottom of the human tongue are covered with taste buds, as are the roof of the mouth and the back of the throat. So administration can be difficult if the composition tastes unpleasant, leading to nonadherence or noncompliance. It is therefore advantageous if an orodispersible formulation has a pleasant taste.

However, having noted that a pleasant taste is necessary for compliance, it is also notable that it is easy for children to mistake CBD-or THC-containing edibles such as gummies or gum drops for candy, as the gummies look good, taste good, and the packages can be bright and colorful. This can be dangerous for children and lead to accidental poisonings. In 2020, the American Association of Poison Control Centers reported more than 3,000 exposures to edible marijuana products involving children 12 and younger in the United States. Therefore, the present invention intends to provide pucks or pellets that are not attractive to children, and which can be stored in child-resistant packaging or containers.

In light of the above discussion, while current products containing nutraceuticals may be useful for their intended purposes, it would be advantageous to provide a freeze-dried fruit puree composition in the form of an orally administered puck or pellet, which has a pleasant taste and rapidly melts in the mouth for absorption into the bloodstream through the oral mucosa. In particular, it would be beneficial to provide a fruit-based, readily dissolvable tablet or melt containing a nutraceutical such as CBD or Δ-8-THC, which has a pleasant taste while also providing fast-onset and improved bioavailability. It would also be useful if such a puck or pellet were shaped in a manner that they are not attractive to children in appearance.

### SUMMARY OF THE INVENTION

Accordingly, the present invention provides freeze-dried, lipid modified, fruit-based edibles in a form that can rapidly melt in the mouth of a subject, such that the nutraceutical or active ingredient is absorbed through the oral mucosa. The composition can include a fruit puree, at least one nutraceutical such as CBD or Δ-8-THC, and an edible oil. In a preferred embodiment an edible wax can be used in combination with the edible oil, and the composition can be freeze-dried and shaped into a consumable form which is capable of melting in the mouth.

A first aspect of the invention provides a freeze-dried puree composition comprising a mixture of: (a) at least 50% by weight of a fruit puree; (b) one or more nutraceuticals for achieving a desired therapeutic effect; and (c) at least 5% by weight of an edible oil, wherein the puree composition is poured into a mold and freeze-dried in the form of a pellet which melts in the mouth.

A second aspect of the invention provides a freeze-dried puree composition comprising a mixture of: (a) at least 50% by weight of a fruit puree; (b) one or more nutraceuticals for achieving a desired therapeutic effect; (c) at least 2% by weight of an edible oil; and (d) at least 0.2% by weight of an edible wax, wherein the puree composition is poured into a mold and freeze-dried in the form of a pellet which melts in the mouth.

A third aspect of the invention provides a process for the preparation of a puree composition in the form of freeze-dried pellets, the process comprising the steps of: (a) adding one or more nutraceuticals to a fruit puree; (b) adding an edible oil to the fruit puree containing the one or more nutraceuticals; (c) mixing the fruit puree, the one or more nutraceuticals, and the edible oil until they are uniform throughout the composition, thereby making a homogeneous puree composition; (d) pouring the puree composition into a mold for forming the puree composition into pellets; and (e) freeze-drying the pellets in the mold containing the puree composition until the water content is less than about 5% by weight.

In addition to the features and examples described above, the nature and advantages of the present invention will be more fully appreciated after reviewing the accompanying drawings, detailed description, and claims.

### BRIEF DESCRIPTION OF THE DRAWINGS

The accompanying drawings illustrate embodiments of the invention and, together with a general description of the invention given above, and the detailed description given below, serve to explain the principles of the invention.

FIG. 1 is a photograph of a freeze-dried, lipid modified, fruit-based edible puck according to one embodiment of the present invention, made in a mold with a THC warning label imprinted on the puck.

FIG. 2 is a photograph of a freeze-dried, lipid modified, fruit-based edible puck according to one embodiment of the present invention, made in a mold having the shape of a mushroom, in which an edible oil was used in the puree composition but not an edible wax.

FIG. 3 is a photograph of a freeze-dried, lipid modified, fruit-based edible puck according to one embodiment of the present invention, made in a mold having the shape of a mushroom, in which both an edible oil and an edible wax were used in the puree composition.

### DETAILED DESCRIPTION OF THE INVENTION

The present invention provides a freeze-dried, lipid modified, fruit-based edible in a form that can rapidly melt in the mouth and be absorbed through the oral mucosa of a subject. The composition can preferably include a fruit puree, at least one supplement/nutraceutical, and an edible oil. In one preferred embodiment an edible wax can be used in combination with the edible oil. The resulting puree composition can be poured into a mold that contains wells which shape the poured puree composition into pellets, pucks, melts and/or tablets, as is known in the art. The mold contents can then be freeze-dried into a consumable form, capable of melting in the mouth.

The term “edible oil” as used herein refers to a fat used in cooking or food preparation which is derived from plant or animal sources safe for human consumption. Non-limiting examples of edible oils include olive oil, coconut oil, palm oil, butter, ghee, vegetable oil, avocado oil, peanut oil, sunflower oil, sesame oil, corn oil, safflower oil, cottonseed oil, and flaxseed oil. Edible oils are a main source of unsaturated fats in human diets.

As used herein, the phrase “edible wax” refers to natural substances used as food additives, typically as coatings and glazing for foods like fruits, vegetables, and candy. Common examples include beeswax, carnauba wax, paraffin wax, candelilla wax, sugarcane wax, rice bran waxes, or other food-grade waxes that are used in the food industry. Such waxes are known to provide shelf-life extension, moisture retention, and a shiny appearance, and are generally considered safe for human consumption. Edible waxes are typically used only in tiny amounts.

As used herein, the term “fruit puree” refers to a thick, smooth, creamy paste or liquid made by blending or mashing cooked (or uncooked) fruits into a smooth consistency. During the cooking process, the fruits soften, and their natural sugars caramelize and concentrate, resulting in a flavorful, slightly thickened sauce. While the purees used for the present invention do not, some fruit purees have sweeteners added to enhance their taste. Fruit purees can be used in a lot of different ways, and can improve the taste of baked goods. These smooth, concentrated fruit mixes naturally add sweetness, flavor, and moisture to muffins, cakes, breads, and other baked goods.

The term “medium-chain triglycerides” or “MCTs” as used herein refers to edible oils that are made in a lab from coconut and palm kernel oils. While typical dietary fats are long-chain triglycerides (LCTs) which have fatty acids with more than 12 carbons, medium-chain triglycerides contain fatty acids with 6-12 carbons. MCTs are absorbed directly into the portal vein and transported to the liver for rapid oxidation, bypassing the need for bile salts or pancreatic lipase for digestion. MCTs are a fat source for people who cannot tolerate other types of fats, such as patients suffering from involuntary weight loss issues, called cachexia or wasting syndrome. MCTs can also be used as emulsifiers, as well as in pharmaceutical preparations, cosmetics, and food applications.

As used herein, the terms “freeze-dried” or “freeze-drying” or “lyophilized” refer to a dehydration technique based on the sublimation of water in a product. The process involves freezing the product and lowering the pressure, thereby removing the ice by sublimation. This is in contrast to dehydration techniques which evaporate the water in a product, using heat. Because of the low temperature used in processing, the rehydrated product retains many of its original qualities. A properly freeze-dried puree according to the invention described herein can have an extended shelf life ranging from several months to several years.

As used herein, the terms “weight percent”, “percent by weight”, or “% by weight” refer to the weight of an ingredient or component divided by the weight of the composition that includes the ingredient, multiplied by 100%. For example, the weight percent of component A when 5 grams of component A is added to 95 grams of component B is 5% (e.g., 5 g A/(5 g A+95 g B)×100%).

The present invention utilizes the technique of freeze-drying, typically of a fruit puree. Purees of strawberries, blueberries, mangos, tangerines, raspberries, bananas, apples, and pineapples are commonly freeze-dried, as is known in the art. Freeze-drying preserves the nutritional content of the food, including vitamins, minerals and antioxidants, more effectively than other preservation methods. The low temperatures used during freeze-drying minimize nutrient loss. Unlike canning or dehydrating, freeze-drying retains more of the original flavors, colors and textures of fresh produce.

According to the present invention, a predetermined amount of fruit puree can be placed in a mixing bowl, then one or more supplements or nutraceuticals of choice, along with a predetermined amount of edible oil (such as medium chain triglyceride (MCT) oil) and/or edible wax, can be added to the fruit puree. The contents of the mixing bowl are then stirred together or otherwise mixed until they are uniform throughout the composition, and the puree mixture is then poured into a mold having wells that can shape or form the poured puree into multiple pellets, pucks, melts and/or tablets. While one or more supplements are typically added directly to the fruit puree along with the edible oil, it may sometimes be advantageous to first dissolve the supplements into the edible oil and/or an edible oil/wax combination, prior to adding them to the fruit puree. For example, when one or more of the nutraceuticals being added to the fruit puree is not readily water or oil soluble (such as THC, which is required to be homogeneous and/or have a uniform distribution in the final product), the nutraceutical can first be dissolved in the edible oil, which is typically done at greater than 50° C. (with or without also first adding an edible wax to the edible oil, as described herein). A similar solution can also be prepared by dissolving the THC in food grade alcohol (ethanol). The resulting nutraceutical and edible oil/wax solution can then be added to the mixing bowl with the fruit puree, and the puree composition (including all ingredients) can then be mixed and poured into the mold for making pellets.

The content or amount of edible oil (such as MCT oil) in the final puree composition can be between about 5% and 40% MCT oil; however, when edible wax such as 2% beeswax is added to the MCT oil to create an oil/wax solution, the amount of edible oil needed for the puree composition may be decreased to between about 2% and 20%. Specifically, the edible wax can be added to the edible oil to create an oil/wax solution, which is then added to the puree composition. The nutraceutical can be also added to the oil/wax solution prior to adding everything to the fruit puree, if needed for homogeneity, or the nutraceutical can be added directly into the fruit puree, depending on the water solubility needs of the nutraceutical, as explained above. If an edible wax is used, the typical concentration of edible wax in the oil/wax solution may be about 2% by weight, such that the concentration of edible wax in the final puree composition may be about 0.2%. In any event, the amounts of edible oil and edible wax can be tailored or otherwise adjusted as needed, for example, to improve product mold release, to provide reliable creation of imprints on the product pellet, as well as to create a more stable pellet for shipping. According to the present invention, the use of an edible wax such as beeswax along with an edible oil such as MCT oil, and adding this in combination with a fruit puree and other supplements, can serve multiple functions: (1) as a binding agent to help the puree conform to the shape of the mold; (2) as an emulsifier to soften the freeze-dried pellet; (3) for successful removal of the product from the mold as a single pellet; and (4) to allow for the reliable creation of imprints or cautionary marks on the pellet.

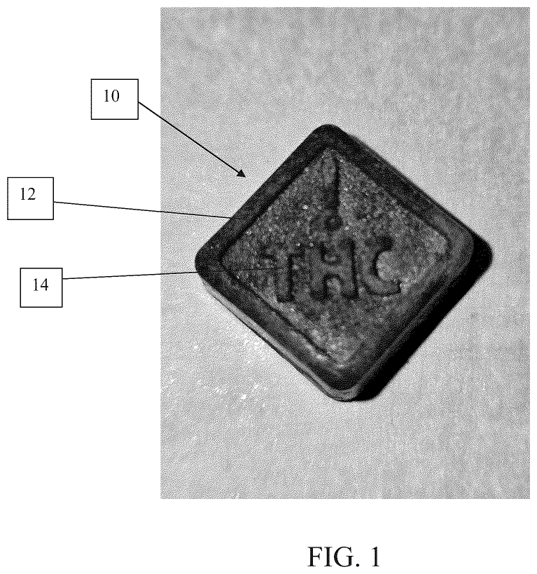

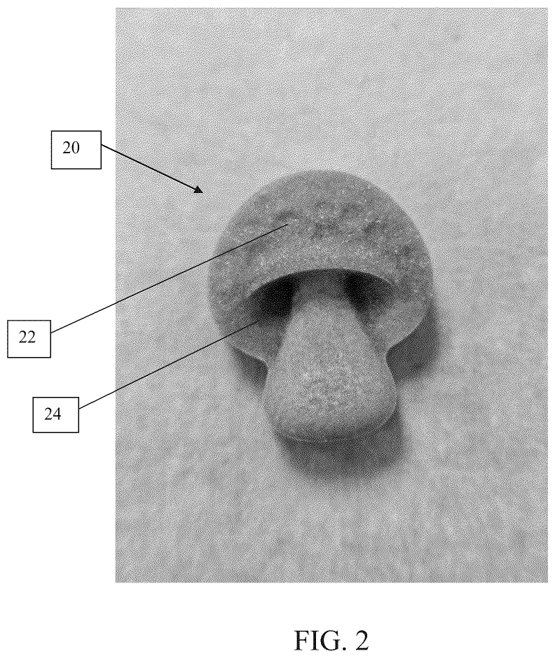

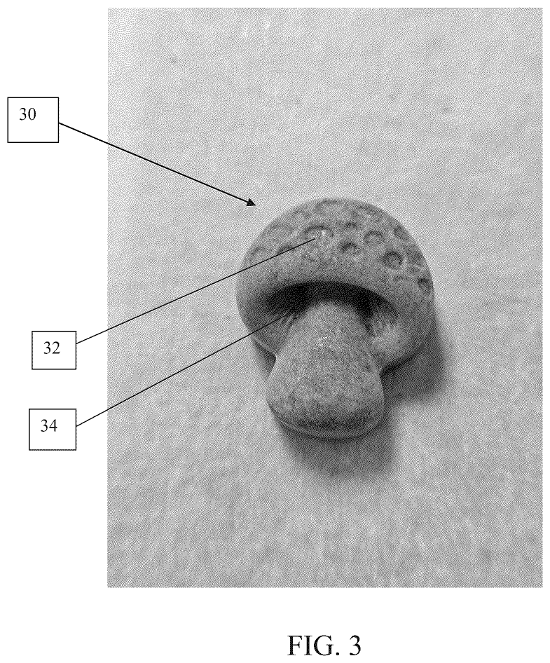

The use of an edible oil such as medium chain triglyceride (MCT) oil with or without the addition of edible wax can impact the speed and ease of the product's release from the mold, and can also influence the detail the product is able to hold from the mold. However, the inventor has observed that the inclusion of an edible wax together with an edible oil greatly improves the ease of release from the mold. The use of an edible wax together with the edible oil can also improve the reliability of producing clearly defined, non-crumbling imprints or cautionary marks which may be embedded on the pellets, seen as letters, numbers, symbols, imprint codes, or labels. Such cautionary marks are often required, and can help with medication identification, dosage, safety, and potential drug interactions. The FDA requires that most oral dosage forms have such an imprint. This is especially important for THC products, where the imprinted designation of the product containing THC is required to be retained, and legible, on the edible product. As seen in FIG. 1, the inventive pellet or puck 10 can be made in a mold to include a border 12 and having a THC warning label 14 imprinted on the puck. FIG. 2 shows a puck 20 according to one embodiment of the present invention, made in a mold having the shape of a mushroom, in which only an edible oil was used in the puree composition, but not an edible wax. The mushroom-shaped puck 20 in FIG. 2 can be viewed in contrast to the much more well-defined mushroom 30 shown in FIG. 3, in which both an edible oil and an edible wax were used in the puree composition. It can be appreciated that the detail on both the outside cap 32 and inside cap 34 of the mushroom 30 in FIG. 3 is much superior to the outside cap 22 and inside cap 24 detail of the mushroom 20 of FIG. 2. It can also be appreciated from viewing the Figures that the product pellets/pucks 10, 20, 30 appear medicinal in nature, and typically would not be readily viewed by children as a type of treat, or candy.

In addition to providing for better detail produced by the mold in the final product, the inclusion of an edible wax together with an edible oil allows for improved durability of the pellet produced, which affects the marketability of the product, as it must stand up to shipping and handling without crumbling. In addition, the lipids present in the edible waxes and oils improves the ability of the product to “melt” in the mouth and adhere to the oral mucosa. The inclusion of an edible wax together with an edible oil also has the effect of allowing more fruit puree to be included in each pellet/puck. For example, the typical fruit puree content of the puree composition can be increased to about 80% when including beeswax with the edible oil, which is an improvement from a maximum of 65% fruit puree when only an edible oil is used. To accomplish this, the edible wax can be dissolved with the edible oil, typically at temperatures above 50° C., and the oil/wax solution is then added to the puree composition along with the fruit puree and at least one nutraceutical such as a cannabinoid and/or other supplements. The mixing of these ingredients into a puree composition is typically done in a mixing bowl at near ambient temperature. In a preferred embodiment, an edible oil/wax solution of approximately 2% beeswax in MCT oil is used. At this concentration the solution remains pourable, and the wax remains dissolved when it is added to the mixing bowl. Since the fruit puree is typically refrigerated, and is the primary component of the mix, oil/wax solutions having higher wax concentrations will typically cause the wax to come out of solution and keep the wax from distributing uniformly in the overall puree composition. This can lead to uneven wax distribution in the pellets/pucks when poured into the molds.

To prepare the inventive pellets/pucks, initially a mixing bowl can be placed on a scale, which is then tared. Taring the scale's display to zero provides an accurate weight of the substances that are next to be added to the mixing bowl, without including the bowl's weight. A predetermined weight of fruit puree, typically purchased in bulk from a wholesaler, can then be poured into the bowl, and the scale can once again be zeroed. Nutraceutical supplements intended to create a desired effect, preferably such as Δ-8-THC and/or CBD (which can be purchased online) are then selected and added to the composition; for example, THC for drowsiness and CBD for pain control prior to falling asleep. Supplements such as Lion's mane, Goji berry, maca root and rhodiola can also be added.

Once one or more nutraceutical substances and/or other supplements are chosen to create a desired effect, they can each be measured precisely and added to the mixing bowl in predetermined amounts, and the scale can be tared after each. Thus the contents and concentration of each element added to the mixing bowl is known. Finally, the desired amount of edible oil, preferably in combination with an edible wax as described above, can be added to the mixing bowl. The composition is then homogenized by being placed in a standard mixer until all of the puree composition's components are distributed uniformly. As noted above, it may be advantageous to first add the nutraceutical to the edible oil or edible oil/wax solution, to ensure homogeneity during mixing. Mixing the puree composition is usually continued for about 10 minutes, then the contents of the bowl can be poured into molds having desired shapes. As noted above, the present invention intends to provide pucks or pellets that are not attractive to children, and which can be stored in child-resistant packaging or containers. The molds used to prepare the pucks/pellets of the present invention, therefore, typically provide shapes that do not look like candy, but rather appear more medicinal in nature.

Silicone molds for shaping a final product can be purchased having standard shapes, such that the products can be in the form of a pellet, a puck, a tablet, various fruit shapes, mushrooms, teardrops, and other geometric shapes. However, many commercial-grade suppliers can also create custom shapes as well. Once the mold is loaded with the puree composition, the mold is then either taken to the freezer for pre-freezing, or put directly into the freeze dryer, where the temperature is gradually lowered and a vacuum is created. The freeze-drying step can be accomplished with small home models, or large commercial units. This process freezes the puree composition within the mold, and initiates sublimation, where water vapor transitions from a solid state to a gas without passing through the liquid phase.

The freeze-dryer then collects and removes the moisture, and once the freeze-drying process is complete, the mold containing the dried puree composition can be removed from the freeze-dryer and the pucks/pellets can then be removed from the molds. The product pucks/pellets can be loaded into containers that are able to endure and sustain a vacuum, for example, airtight containers such as glass mason jars, or 5-gallon containers designed for vacuum storage and to protect against moisture and air causing spoilage. This is an intermediary holding step to provide a buffer between production and packaging. When the product is ready to be packaged and shipped, it is put into plastic containers with child-resistant, tamper-evident lids, then labeled, dated for easy identification, and boxed for shipping.

The amount of edible oil that is added to the puree composition can vary depending on the capacity of the specific fruit puree being used to accept the oil. Strawberry is an example of a fruit puree with a relatively low oil requirement, typically about 10-15% by weight. However, this value may be higher for more viscous purees, such as peach and mango, in which MCT oil is typically added at between about 20% and 25% by weight of the wet mixture. Nevertheless, as noted above, it has been found that the addition of an edible wax such as beeswax lowers the amount of edible oil needed. For example, a strawberry pellet/puck with both edible oil and edible wax in the puree composition can be prepared that melts rapidly in the mouth, is easy to remove from the mold, and is stable for shipping, with about 5% by weight of MCT in the wet mix. Therefore, the use of an edible wax decreases the edible oil requirement by about half. Mango produces a satisfactory product at about 10-12% by weight when both an edible oil and an edible wax are included in the puree composition.

As noted above, the lipid modification produced by adding both an edible oil and an edible wax can be advantageous. The lipophilicity of the added edible wax can cause the product pellet/puck to dissolve more rapidly in the mouth, providing faster delivery and enhanced bioavailability of the lipophilic cannabinoids and supplements, via enhanced absorption through the oral mucosa. In addition, as noted above, an edible oil such as MCT oil in combination with and edible wax such as beeswax also aids in release of the pellets from the mold at the completion of the freeze-drying step, and especially aids with the ability of the mold to create legible cautionary marks on the pellets, such as letters, numbers, symbols, or imprint codes, which can help with medication identification.

The final product, typically in the form of pucks or pellets made according to the present invention, can also be designed to produce different results by tailoring the type of fruit puree used to the desired effect. Specifically, certain fruits, vegetables, flowers and herbs can be selected for their flavor and functionality, such as to provide functional terpenes that guide what is referred to herein as the “entourage effect”, which is an enhancement of the final product. For examples, certain fruit purees may enhance drowsiness, while others may enhance wakefulness. Different fruits, or combinations of fruits can contain different functional chemicals that can influence human behavior. All fruits get their aroma, color, and flavor from naturally existing terpenes or terpenoid compounds. Common terpenes include myrcene, limonene, caryophyllene, pinene, linalool, terpinolene, ocimene, and humulene

For example, it is envisioned that a combination of fruit purees can be selected according to their terpene content to produce a drowsy effect, while others may be more suitable to be taken in the morning, producing mood elevation, stress relief, and wakefulness. By choosing purees with stronger flavors, such as black currant or passion fruit, or by selecting fruit purees with better covering flavors, different desired effects can be generated in the final product. Black currants are a type of berry native to temperate parts of Europe and Asia, known for their tart, sweet flavor and high vitamin and antioxidant content, often used in jams, juices, and other culinary applications. Passion fruit is a tropical fruit, known for its sweet and tart flavor, with notes of citrus, melon, and pineapple. Mangos are known for their sweet, aromatic, and juicy flavor and contain myrcene, which has potential sedative and relaxing effects, and allows psychoactive chemicals such as Δ-8-THC and/or CBD better access across the blood/brain barrier. A cherry puree contains terpenes that can produce an anti-inflammatory effect, and blueberries have lavender terpenes which can impart a calming effect and drowsiness. Strawberry purees are also calming, and tangerines are useful when taken in the morning because they include certain terpenes which can elevate mood and create a wakeful effect. Vegetable purees such as beets may also be considered. Indeed, it is envisioned that the same puree process used herein for fruits can be extended to vegetable purees, tuber purees, meat purees, as well as coffee, or other hot/cold drinks. For example, desired nutraceutical supplements can be added to a coffee puree composition, so that one or more pellets of the product can be dissolved in a cup of hot water to make instant, nutraceutical-enhanced coffee.

Other supplements or nutraceuticals can be added to the puree composition as well. For example, an anxiolytic such as Lion's Mane extract has been found to be useful in appropriate doses in combination with various fruit purees. Lion's mane is a fungus extract with a mild, slightly sweet flavor which can improve cognitive function, boost the immune system, improve mood, and may have anti-diabetic, anti-inflammatory and antioxidant properties. Other fungal extracts are less desirable for use with the present invention, as they often have a strong woody or “earthy” taste. Nevertheless, Turkey Tail and Cordyceps fungi extract powders have been used in coffee puree preparations done according to the present invention. Goji berry extract can also be added, which is available as a powder from the fruit of the Goji plant, and is very high in antioxidants and considered to contain reproductive or aphrodisiac properties. Maca root has been traditionally used as an aphrodisiac, and is thought to help regulate hormones, particularly in women going through menopause. It may help reduce hot flashes, mood swings, and other symptoms. Some studies suggest that it may improve sexual desire and function in both men and women. Rhodiola can be added to the product composition to increase energy levels and endurance, improve athletic performance, reduce fatigue, and may regulate blood sugar levels.

The present invention uses hemp derived Δ-8-THC and CBD as the preferred cannabinoid nutraceuticals. Δ-8-THC can be legally produced by growing hemp, and it is known to “hit” much more subtly than Δ-9-THC. The invention also specifically uses Δ-8-THC distillates or concentrates, which can be purchased on the market. While natural terpenes are abundant in cannabis plants, terpenes are not present in Δ-8-THC distillates or concentrates. However, the lack of terpenes in Δ-8-THC concentrates is not an issue with the present invention since, as noted above, the invention relies upon terpenes present in the various fruit purees to provide the “entourage effect”, rather than native marijuana terpenes.

In addition to using concentrated Δ-8-THC and CBD as the added nutraceutical, the present invention also envisions incorporating other THC/CBD variants, such as cannabigerol (CBG), cannabinol (CBN), tetrahydrocannabivarin (THCV), Cannabichromene (CBC), cannabivarin (CBDV), Delta-9-tetrahydrocannabiphoral (THCP), cannabidiphorol (CBDP), Δ-6-THC, Δ-10-THC, Cannabidiolic acid (CBDA), Tetrahydrocannabinolic acid (THCA), 11-hydroxy-Δ-9-THC, levonantradol, and others.

According to the present invention, the amount of fruit puree in the final puree composition is typically between about 50% by weight and about 95% by weight. Certain preferred nutraceuticals, listed herein, are intended to be added to the puree composition so that each product pellet/puck contains an amount or dosage, typically in milligrams (mg), which is capable of achieving a desired therapeutic effect. This can include between about 1 mg and about 25 mg of Δ-8-THC, preferably between about 2 mg and 10 mg. CBD typically requires a higher dosage than THC, for example, commercial dosages for THC edibles are generally 2 mg-10 mg per dose (i.e., in a single pellet or other type of edible, such as a gummy), while CBD is typically 10 mg-20 mg per dose.

According to some embodiments of the invention, the puree composition may include between about 5% by weight and 40% by weight MCT oil per dose; however, as noted above, when edible wax such as 2% beeswax is added to the edible oil to create an oil/wax solution, the amount of edible oil needed for the puree composition may be decreased to between about 2% and 20%, preferably between about 5% and 15%, and more preferably about 10%, when combined with edible wax in an oil/wax solution. The typical concentration of edible wax in the oil/wax solution may be about 2% by weight, such that the concentration of edible wax in the final puree composition can be about 0.2%. According to some embodiments, the composition may also include between about 0.1 g and 1.0 g of Lion's mane, which corresponds to between about 2% by weight and about 20% by weight, of the puree composition. According to some embodiments, the composition may also include between about 0.1 mg and about 0.5 mg of Gogi berry per dose, which corresponds to between about 2% and about 10% by weight of the wet puree composition.

As described herein, the present invention provides a process for the preparation of a freeze-dried, fruit-based composition which includes mixing together a fruit puree and a supplement or nutraceutical (such as Δ-8-THC and/or CBD) with a specific amount of edible oil or edible oil/edible wax combination. As noted, the edible oil helps to aid in binding of the composition together as well as easy release from the mold. The addition of edible wax improves these qualities of binding and easy release. Lion's mane, Gogi berry, or other nutraceuticals can also be added to the fruit puree composition prior to homogenization, and then the puree composition is typically poured into a mold for making pellets. The composition within the mold is then freeze-dried under suitable conditions, until water content is reduced to an amount which can create a ready to consume nutraceutical product which melts in the mouth. Typical water contents achieved in freeze drying are below 5% by weight. This value can be adjusted, but the low water content is useful to prevent mold and fungal growth that can normally occur on a sugar-containing product like fruit.

The following examples are presented in order to more fully illustrate some embodiments of the invention. They should in no way be construed, however, as limiting the scope of the invention, and one skilled in the art can readily devise many variations and modifications of the principles disclosed herein without departing from the scope of the invention.

EXAMPLES: Example 1: a puree composition was created including a blueberry puree, 5 mg Δ-8-THC, MCT oil 20% by weight, and 0.5 g Lion's mane. The product pellets/pucks generated from this composition are useful for stress reduction, as a sleep aid, and for general relaxation.

Example 2: a puree composition was created including a tangerine/mango puree mix, 10 mg CBD, an MCT/beeswax solution 10% by weight, 0.5 g Lion's Mane, and 0.25 g Gogi berry. The product pellets/pucks generated from this composition are useful for a morning energetic effect, or for a calming effect, as well as for immune support.

Example 3: a puree composition was created including a strawberry puree, 10 mg Δ-8-THC/CBN mix, and an MCT/beeswax solution 10% by weight. The product pellets/pucks generated from this composition are useful as a highly drowsy mix for people with more difficulty sleeping.

Example 4: a puree composition was created including a cherry puree, 5 mg Δ-8 -THC, an MCT/beeswax solution 10% by weight, 0.5 g Goji Berry, and 2 g maca root. The product pellets/pucks generated from this composition are useful for immune support, to reduce inflammation, and alleviate menopausal symptoms, as well as being a potential aphrodisiac.

Products manufactured according to the various embodiments of the invention presented herein, while capable of being dissolved in water prior to use, are intended to be “ready to eat” products, such that they do not have to be rehydrated, steamed, cooked, dissolved, microwaved or further prepared in any manner. The invention does not consider the use of artificial sweeteners or flavoring agents.

While particular embodiments of the present invention have been illustrated and described herein in considerable detail, such embodiments are not intended to restrict or limit the scope of the appended claims. Accordingly, it should be understood that additional modifications and variations of these embodiments, and the equivalents thereof, are within the scope of the invention as recited in the following claims.

What is claimed is:

1. A freeze-dried puree composition comprising a mixture of:
a) at least 50% by weight of a fruit puree;
b) one or more nutraceuticals for achieving a desired therapeutic effect; and
c) at least 5% by weight of an edible oil, wherein the puree composition is poured into a mold and freeze-dried in the form of a pellet which melts in the mouth.

2. The composition of claim 1, further comprising an edible wax, wherein the edible wax in combination with the edible oil aids in the formation of the pellet within the mold and the release of the pellet from the mold, and wherein the edible wax can decrease the required amount of edible oil.

3. The composition of claim 2, wherein the edible wax comprises about 0.2% of the puree composition, and wherein the edible oil comprises between 2% and 20% of the puree composition.

4. The composition of claim 1, wherein the fruit puree comprises between about 50% by weight to about 95% by weight of the composition.

5. The composition of claim 1, wherein the edible oil comprises between about 5% by weight to about 40% by weight of the composition.

6. The composition of claim 1, wherein Δ-8-THC is one of the one or more nutraceuticals, and wherein each freeze-dried pellet includes between 1 mg and 10 mg of Δ-8-THC.

7. The composition of claim 1, wherein CBD is one of the one or more nutraceuticals, and wherein each freeze-dried pellet includes between 10 mg and 25 mg of CBD.

8. The composition of claim 1, wherein Lion's mane is one of the one or more nutraceuticals, and wherein Lion's mane comprises between 2% by weight and 20% by weight of the puree composition.

9. The composition of claim 1, wherein Gogi berry is one of the one or more nutraceuticals, and wherein Gogi berry comprises between 1% by weight and 10% by weight of the puree composition.

10. The composition of claim 1, wherein Maca root is one of the one or more nutraceuticals, and wherein Maca root comprises between 1% by weight and 20% by weight of the puree composition.

11. The composition of claim 1, wherein the fruit puree is selected from the group consisting of strawberries, blueberries, mangos, tangerines, raspberries, bananas, apples, black currant, passion fruit, and pineapples.

12. The composition of claim 1, wherein the one or more nutraceuticals are selected from the group consisting of Δ-8-THC, CBD, Lion's mane, Goji berry, maca root and rhodiola.

13. The composition of claim 1, wherein the edible oil is selected from the group consisting of olive oil, coconut oil, palm oil, butter, ghee, vegetable oil, avocado oil, peanut oil, sunflower oil, sesame oil, corn oil, safflower oil, cottonseed oil, and flaxseed oil.

14. The composition of claim 2, wherein the edible wax is selected from the group consisting of beeswax, carnauba wax, paraffin wax, candelilla wax, sugarcane wax, rice bran waxes, and other food-grade waxes that are used in the food industry.

15. A freeze-dried puree composition comprising a mixture of:
a) at least 50% by weight of a fruit puree;
b) one or more nutraceuticals for achieving a desired therapeutic effect;
c) at least 2% by weight of an edible oil; and
d) at least 0.2% by weight of an edible wax, wherein the puree composition is poured into a mold and freeze-dried in the form of a pellet which melts in the mouth.

16. A process for the preparation of a puree composition in the form of freeze-dried pellets, the process comprising the steps of:
a) adding one or more nutraceuticals to a fruit puree;
b) adding an edible oil to the fruit puree containing the one or more nutraceuticals;
c) mixing the fruit puree, the one or more nutraceuticals, and the edible oil until they are uniform throughout the composition, thereby making a homogeneous puree composition;
d) pouring the puree composition into a mold for forming the puree composition into pellets; and
e) freeze-drying the pellets in the mold containing the puree composition until the water content is less than about 5% by weight.

17. The process of claim 16, further comprising, following the step of adding an edible oil, adding an edible wax to the fruit puree containing the edible oil and the one or more nutraceuticals, wherein the edible wax in combination with the edible oil aids in the formation and release of the pellet from the mold, and wherein the edible wax can decrease the required amount of edible oil.

18. The process of claim 16, wherein the process provides a plurality of pellets, each pellet comprising a homogeneous mixture of freeze-dried puree composition which melts in the mouth.

19. The composition of claim 1, wherein a coffee puree is substituted for the fruit puree, and wherein one or more pellets are dissolvable in a cup of hot water for drinking.
